(19) United States
(12) Patent Application Publication
(10) Pub. No.: US 2025/0147473 A1
(43) Pub. Date: May 8, 2025

(54) INTEGRATED GAS SENSING AND REGULATION SYSTEM

(71) Applicant: Prolec-GE Waukesha, Inc., Waukesha, WI (US)

(72) Inventors: John Pruente, Waukesha, WI (US); Arthur Martin, Waukesha, WI (US); Danny Mathew, Waukesha, WI (US)

(21) Appl. No.: 18/933,158

(22) Filed: Oct. 31, 2024

Related U.S. Application Data
(60) Provisional application No. 63/595,926, filed on Nov. 3, 2023.

(51) Int. Cl. G05B 13/04; G01L 19/00; G01N 33/00; H01F 27/20

(52) U.S. Cl. CPC G05B 13/04; G01L 19/0092; G01N 33/005; H01F 27/20

(57) ABSTRACT

An enhanced N₂ regulation system is integrated with a H₂ sensor together as a combined package. This system is designed to detect the generation of hydrogen within a transformer, directly within the nitrogen headspace of the transformer. The present invention also ensures a system's operation that maintains the set pressure range by either adding nitrogen or venting overpressure to the atmosphere as required. The final stage of the N₂ regulator system is responsible for maintaining the gas-blanketed tank pressure within the range of 0.5 to 5.0 psi consistently.

### CROSS REFERENCE TO RELATED APPLICATION

This application claims priority to U.S. Provisional Application No. 63/595,926, filed Nov. 3, 2023, the disclosure of which is hereby incorporated by reference in its entirety.

### FIELD OF THE INVENTION

The present invention is related to an enhanced N₂ monitoring system by integrating it with a H₂ sensor together as a combined package for early detection and warning of potential faults in a nitrogen-blanked transformer. The present invention provides a more efficient way to prevent transformer failure and prepare for maintenance. Furthermore, it is a cost-effective solution with the ability to remotely diagnose abnormal conditions in transformer oil.

More specifically, the present invention is applicable to dielectric mineral oil filled Transformer with a nitrogen blanket oil PRESERVATION system.

### BACKGROUND OF THE INVENTION

The slow response to developing faults in oil-filled nitrogen-blanketed transformers can lead to unplanned and costly downtime. Further, the late detection of hydrogen levels in the transformer, typically done through annual oil sampling, can delay the identification of potential issues. So, this insufficient or intermittent monitoring can make it difficult to identify incipient faults that could lead to catastrophic failures.

The known prior art discloses sensing apparatus so as corresponding methods thereof, such as patent KR102598373B1, issued on Nov. 3, 2023, discloses a system designed for monitoring distribution transformers. This system includes a monitoring unit, referred to as monitoring unit 102, which is responsible for assessing fluid quality, fluid levels, and tank pressure within the transformer. The monitoring unit 102 checks the quantity of insulating fluid present in the transformer to ensure that no active parts are left without adequate fluid coverage. It generates alerts when abnormal or critical fluid levels are detected. Additionally, the unit monitors the internal pressure of the distribution transformer 106, an important parameter for identifying potential internal fault conditions, and triggers alarms for any abnormal or critical fluid or gas space pressures.

Monitoring unit 102 is also equipped to analyze dissolved gases within the insulating fluid, such as hydrogen, oxygen, nitrogen, carbon monoxide, carbon dioxide, methane, ethane, ethylene, acetylene, propane, and propylene. The presence of these gases may signal aging or dielectric issues within the transformer. The monitoring unit 102 is capable of collecting and analyzing other sensor data indicative of internal transformer conditions, such as sound emissions and vibrations. It can also monitor the status of protection fuses associated with the transformer.

To facilitate remote monitoring, at block 1430, the monitoring unit 102 utilizes a communications unit 104 to transmit sensor data and other generated information to remote devices. This data encompasses real-time sensor measurements, configuration data, transformer nameplate information, and historical trends. The communication can occur via wireless protocols such as Wi-Fi and Bluetooth, or through a wired connection. The remote devices can include computers, servers, tablets, smartphones, or other devices compatible with the monitoring unit's data.

The monitoring unit 102 can generate alarms or notifications based on the analyzed sensor data and can control external components, such as load break switches. It functions as a trigger for user protection systems in various contexts, such as substations. Furthermore, the system can convey identification data related to the transformer, including nameplate information, test reports, and GPS location information for maintenance personnel to easily locate the transformer during fault conditions.

The assembly of the monitoring unit is achieved through a method that includes creating an opening in the transformer tank, attaching a flange around the opening, and inserting a sensor probe array into the tank. The method concludes with securing the monitoring unit to the flange. The entire monitoring system is designed for adaptability and can be retrofitted to existing transformers. The nature of the monitoring capabilities allows for continuous oversight of the transformer's condition, in an effort directed to perform maintenance and operational decision-making based on the data collected.

Furthermore, U.S. Pat. No. 9,234,834B2, issued on Jun. 26, 2014 discloses a system for measuring the water and hydrogen content in insulating oil, particularly used in transformers. This system employs two light sources, specifically radiation sources 60a and 60b, that emit electromagnetic radiation at different wavelengths to assess the absorption characteristics of water and oil in the medium. The water absorption curve is characterized by several peaks in the absorption spectrum, which indicate a strong wavelength dependency. In contrast, the absorption curve for oil components displays less pronounced wavelength dependence and is typically represented as relatively flat for simplicity.

U.S. Pat. No. 9,234,834B2 outlines a method to reliably measure the water content by analyzing the absorption measurements obtained from the two light sources. It assumes that the oil absorption does not significantly depend on wavelength and proposes a mathematical relationship to extract the water content from the measured values. The light sources are activated alternately or at different times to allow for accurate data collection and calibration of the measurements.

In terms of construction, the system includes optical connections to both water detection and hydrogen detection sections, allowing for a dual assessment of the content present in the insulating oil. The use of standard telecommunications components for the light sources ensures that the system remains cost-effective while maintaining operational reliability. The design also accommodates temperature variations and other parameters that may affect optical properties, suggesting that it is adaptable for different operating conditions.

Moreover, U.S. Pat. No. 9,234,834B2 describes a mechanism for maintaining the integrity of the measurements over time. This involves the use of a heater within the hydrogen detection section to manage the temperature and prevent potential inaccuracies caused by fluctuating environmental conditions. The system incorporates a thermostat-controlled heater and a thermometer to monitor the temperature of both the sensing layer and the insulating oil. This allows for the calculation of hydrogen and moisture content based on solubility factors at the measured temperatures.

In addition to measuring water and hydrogen content, the system is designed to perform health checks on the sensor assembly, providing insights into the performance and potential degradation of the sensing components over time. This feature contributes to the overall reliability of the monitoring system by allowing for early detection of potential issues, which is crucial for maintaining the integrity of electrical equipment.

The measurements can be taken in various operational modes, depending on the functionality of the light sources. If one source fails, the system retains the capability to perform assessments using the remaining source, ensuring continuous monitoring of critical parameters. Furthermore, pre-calibrated data can be employed to estimate the water content solely from measurements using the primary light source if necessary.

On the other hand, U.S. Pat. No. 10,732,164, issued on Jun. 18, 2020 refers to a diagnostic system designed for monitoring the condition of power transformers. The system is capable of evaluating the status of several key subsystems within the transformer. One of its main functionalities includes monitoring partial discharges within the transformer tank, which enables the early detection of insulation issues. This feature offers a more immediate response to defects compared to traditional methods that analyze dissolved gases in the transformer oil.

U.S. Pat. No. 10,732,164B2 incorporates a built-in partial discharge registration system that also assesses the insulation condition of high-voltage bushings. This aspect is crucial given the increasing use of hard-insulated bushings, where even minor levels of partial discharge can indicate potential defects. The system operates within the ultrahigh-frequency (UHF) range, specifically between 0.5 to 1.5 GHz. This frequency range helps to mitigate the effects of low-frequency corona discharges and utilizes the transformer tank as a Faraday cage, thereby reducing interference from external electromagnetic sources.

In addition to monitoring partial discharges, U.S. Pat. No. 10,732,164B2 features a capability for moisture content analysis in the transformer oil. This monitoring system detects water presence in the oil, assessing its concentration due to the detrimental effects that water can have on the electrical strength of the oil. The system employs an adaptive mathematical model to describe the moisture transition processes between the oil and the solid insulation, which is particularly relevant for managing transformer operation under varying loads and temperatures.

Moreover, the system includes temperature monitoring functionalities, with the tank temperature serving as a critical operational parameter. The temperature data is utilized not only for evaluating operational modes but also for various diagnostic models within the expert system. The combination of temperature readings from different sensors allows for comprehensive assessments of the transformer's health.

U.S. Pat. No. 10,732,164B2 also encompasses the ability to measure hydrogen concentration in transformer oil. The specifications outline a range of 25 to 5000 parts per million (ppm) for hydrogen detection, along with a stated accuracy and repeatability for the measurements. The system is designed to function independently or in conjunction with other sensor modules, allowing for flexibility in its application.

The invention specifies technical details such as operational voltage ranges, power consumption limits, and physical dimensions. It emphasizes compatibility with existing infrastructure while providing various communication interfaces, including RS-485 MODBUS RTU, for data transmission and integration into broader monitoring frameworks. The design of U.S. Pat. No. 10,732,164B2 facilitates easy retrofitting on older transformers, enhancing adaptability and operational relevance without altering existing operational paradigms.

Furthermore, US Patent Application No. 20220128539 A1, issued on Apr. 28, 2022, discloses a monitoring system designed to assess the condition of transformers, particularly focusing on various operational parameters that influence their performance and safety. The system integrates multiple sensors that collect data related to vibrations, acoustic noise, oil state, and temperature fluctuations, which are crucial for maintaining the structural integrity and efficiency of transformers.

Central to the invention is the processor, configured by firmware, which tracks vibrations and acoustic noise emanating from the transformer. It correlates this noise with load changes and temperature variations, both absolute and ambient. Through frequency analysis, the processor differentiates between magnetic field-driven vibrations and other vibration types, enabling a detailed understanding of the operational state of the transformer. The processor also utilizes remote processing techniques to establish a “fingerprint” of the transformer's relative age, comparing it with a database of similar transformers.

The system includes flowchart procedures to evaluate the mechanical condition of the transformer, which involve several boxes outlining specific operational steps. In certain scenarios, alarms are raised if the mechanical state is deemed dangerous, or if ingress events are detected, indicating potential issues that require immediate attention.

US 20220128539A1 also addresses the chemical state of the oil within the transformer, as it is critical to maintaining insulation breakdown strength. The processor uses data from hydrogen sensors and moisture sensors, calibrated with temperature readings, to assess the oil's condition. High moisture levels in the oil can significantly reduce breakdown strength, necessitating continuous monitoring of water activity over time to detect potential issues.

The invention further discusses the dynamics of moisture movement within the transformer, noting that temperature changes can drive moisture in and out of the insulation. This process is slower compared to rapid temperature fluctuations, which can complicate moisture assessments at any given time.

Dissolved Gas Analysis (DGA) is highlighted as a conventional method for transformer condition monitoring, though it is often hindered by sampling inaccuracies. The invention aims to enhance the accuracy of DGA results by correlating sample data with real-time measurements of hydrogen gas concentration, water activity, and temperature.

The sensor assembly can be connected to a remote server, enabling data transmission for further analysis. This allows for continuous monitoring and assessment of transformer conditions, potentially leading to more informed maintenance and operational decisions.

The system's design also takes into account the physical constraints of the transformer, incorporating materials that facilitate sensor integration without interfering with the transformer's magnetic fields. The sensors are housed in a probe that can be inserted through the transformer tank, ensuring that critical measurements can be taken without compromising the transformer's integrity.

Patent U.S. Pat. No. 9,977,006, published on Aug. 11, 2016, teaches about an hydrogen sensor devices designed for measuring the concentration of dissolved hydrogen gas in various liquids. The hydrogen sensor devices, specifically labeled as devices 100 and 200, can be inserted into the liquid for direct measurement or installed in a container that holds the liquid. The sensor is equipped with a gas separation film that interacts with the liquid to facilitate the measurement process. The device's configuration allows for ease of attachment and detachment from the container, enabling maintenance activities such as repairs or replacements without disrupting the liquid environment.

The design includes an opening portion on the container to which the hydrogen sensor device is affixed, ensuring that the gas separation film makes contact with the liquid for effective measurement. This setup incorporates an opening and closing valve that prevents liquid spillage while allowing the sensor to take measurements when required. The hydrogen sensor device can be coupled to the container in a manner that ensures both gaseous and liquid sealing, enhancing its operational reliability. Various methods for fastening the sensor to the container are described, including threaded connections and alternative fastening mechanisms.

In addition, the hydrogen sensor device is engineered to measure hydrogen concentration continuously or periodically, which is particularly useful for monitoring the condition of oils in mechanical devices, such as transformer oil. The sensor can be installed in such a manner that it remains in place for ongoing assessments without the need for frequent repositioning. Depending on the design configuration, the opening for the sensor can be positioned at different locations on the container, such as the top or side.

U.S. Pat. No. 9,977,006B2 also details an integrated control device that governs the operations of the sensor unit. This control unit includes temperature and liquid inflow sensors, facilitating real-time monitoring of environmental conditions around the sensor. The control unit manages the heater element, ensuring that the sensor reaches an optimal measurement temperature before initiating hydrogen concentration measurements. The control device is capable of analyzing various parameters, including the concentration of oxygen in the sealing space, and can execute appropriate actions based on predetermined thresholds.

The measurement methodology outlined in U.S. Pat. No. 9,977,006B2 incorporates several steps, including temperature measurement, control of the sensor unit's heating mechanism, and the determination of oxygen gas concentration. The process culminates in transmitting the hydrogen concentration data via wired or wireless means for further analysis or display. The apparatus is designed to accommodate various measurement scenarios and can be adapted to suit different liquid types, ensuring versatility in application.

Additionally, U.S. Pat. No. 7,762,121, issued on May 28, 2009 discloses a hydrogen sensor utilizing palladium-silver alloy thin films, arrays, or nanostructures for detecting hydrogen concentrations. The sensor is built on a silicon substrate with a layer of silicon nitride (SiNx) or silicon dioxide (SiO2), which serves as a base for further metal deposition. The invention incorporates various methods for producing palladium-silver nanoparticles or nanowires on the substrate, which play a crucial role in the sensor's functionality.

In one embodiment, a 5000 Å layer of SiNx is deposited onto a silicon substrate, followed by a 1000 Å thick titanium layer. Through lithography, a specific pattern is created on the substrate, and palladium-silver nanoparticles are electroplated onto the titanium layer. The process includes oxidizing the titanium to form titanium dioxide (TiO2) to enhance the sensor's conductive properties. The resulting hydrogen sensor operates effectively at room temperature and has been tested at elevated temperatures, demonstrating functionality up to 178° C.

The device is capable of detecting hydrogen concentrations as low as 0.25% within nitrogen environments. It exhibits varying current responses when exposed to different hydrogen concentrations, indicating sensitivity to hydrogen presence. Notably, the sensor demonstrates a significant current change between the off state (no hydrogen) and the on state (with hydrogen), with variations indicating potential degradation at higher temperatures.

Additionally, U.S. Pat. No. 7,762,121B2 outlines alternative embodiments that enhance the versatility of the sensor design. One such embodiment employs a side-wall plating technique for palladium-silver deposition, allowing for selective growth of nanostructures. This technique minimizes unwanted deposition on the substrate, resulting in a more controlled formation of the sensing elements.

The invention also includes various applications for the hydrogen sensor. These applications range from monitoring hydrogen levels within vehicles and fuel cells to ensuring safety in residential settings and power equipment. The sensor's ability to detect hydrogen buildup in transformer oil is particularly relevant in predicting potential failures in electrical equipment.

Overall, the embodiments of U.S. Pat. No. 7,762,121B2 provide a framework for hydrogen sensors that can be fabricated without relying on transfer methods, which are often associated with degradation in high-temperature environments. The integration of palladium-silver alloy structures on titanium or TiO2 surfaces contributes to the overall performance of the sensor, facilitating the detection of hydrogen at low concentrations while operating effectively across a range of temperatures. The construction and methods detailed in this patent lay a foundation for future developments in hydrogen sensing technology.

Based on all the above, it is clear that within the current landscape of transformer maintenance and monitoring exists a significant gap in the ability to detect hydrogen gas generation effectively and efficiently. Hydrogen is recognized as one of the critical gases indicative of internal faults within transformers, typically arising when the operational temperature exceeds 100° C. The conventional methods for monitoring gas levels necessitate taking transformers out of service for maintenance, leading to costly downtimes and potential catastrophic failures. Additionally, existing oil sampling techniques may only identify the presence of hydrogen as a byproduct while failing to detect other significant gases that can signal underlying issues.

Furthermore, current technologies often rely on the expertise of specialists to assess transformer conditions, rendering the monitoring process less accessible to operators. This reliance underscores a pressing need for solutions that empower users to conduct self-monitoring without requiring specialized training or equipment.

Moreover, the limitations of existing hydrogen sensors further exacerbate these issues. Traditional sensors tend to be standalone devices, often complicated to install and requiring extensive maintenance. The integration of hydrogen detection capabilities within the existing nitrogen regulation systems is not typically achieved, resulting in additional costs and logistical challenges.

There is also a demand for enhanced monitoring capabilities that provide real-time data and predictive maintenance insights. Operators require solutions that not only alert them to the presence of hydrogen gas but also offer remote access to historical data, allowing for trend analysis and informed decision-making.

In summary, the prevailing challenges surrounding hydrogen detection and transformer monitoring highlight a clear need for a comprehensive, integrated solution. The invention presented in this application addresses these deficiencies by providing an efficient, cost-effective method to monitor hydrogen levels directly within nitrogen-blanketed transformers. This innovation aims to enhance operational efficiency, reduce maintenance downtime, and ultimately safeguard transformer reliability.

### SUMMARY OF THE INVENTION

To overcome the deficiencies of the prior art, the present invention provides a predictive maintenance tool designed to keep transformers operating efficiently and prevent major catastrophes. It offers an easy and effective method for monitoring hydrogen (H₂) levels in nitrogen-blanketed transformers. Integrated into the nitrogen (N₂) regulator system and connected to the transformer headspace via the return port of the N₂ regulator, it provides an early warning of combustible gas generation in the transformer, which drive to an efficient risk condition or issue detection, advantageous reducing potential scenarios wherein the transformer could be damaged and consequently drawn off from operation, so as, for prompt detection of boundary conditions and the user could perform any corrective action without interrupting the operation of the transformer.

In a preferred embodiment, the installation of the H₂ sensor is a breeze as it comes as part of the N₂ system, requiring no additional fittings or hardware. This simplifies the installation process and reduces costs. The sensor is self-calibrating and requires minimal maintenance, making use of field-proven technology for reliability.

Another objective of the present invention is to offer remote access to logged data. It provides real-time analog data remotely and offers up to 1 year of data storage for trend analysis. With a sensor life of over 10 years, it is possible to count on long-lasting performance and peace of mind in the transformer monitoring needs.

In a preferred embodiment the present invention refers to an enhanced N₂ regulation system by integrating it with a H₂ sensor together as a combined package. This system is designed to detect the generation of hydrogen within a transformer, directly within the nitrogen headspace of the transformer. The present invention also ensures a system's operation that maintains the set pressure range by either adding nitrogen or venting overpressure to the atmosphere as required.

These and other objectives will be apparent to the person skilled in the technical field to which the present invention pertains, based on the following detailed disclosure, the accompanying figures, as well as the appended claims.

### BRIEF DESCRIPTION OF THE DRAWINGS

Having described the invention in the above general terms, reference will now be made to the accompanying drawings showing representative embodiments of the present invention, where:

FIG. 1 illustrates the block diagram of the Nitrogen (N₂) System with Integrated H₂ Sensor of the present invention so as the specific parts thereof;

FIG. 2 illustrates the Nitrogen system cabinet with hydrogen sensor according to the present invention, and according to a preferred embodiment thereof;

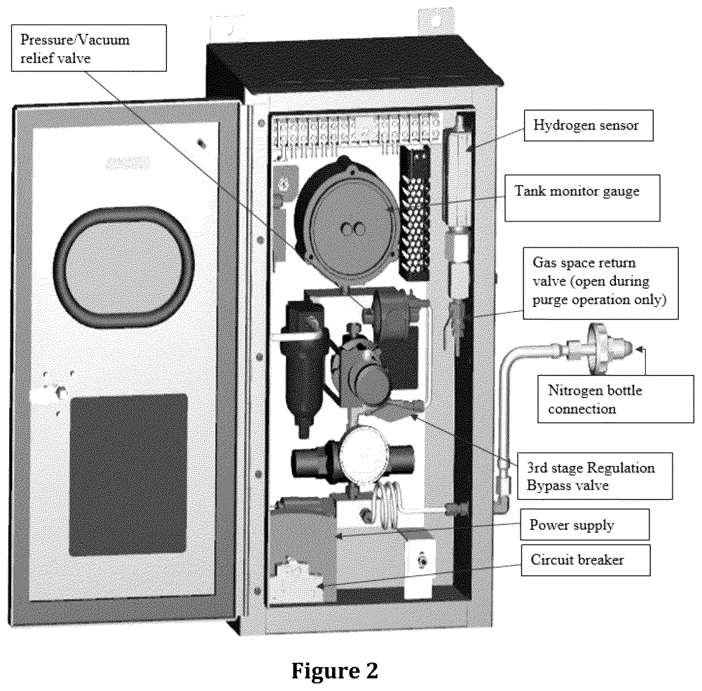

FIG. 3 illustrates a general test setup diagram showing the features of the present invention according to an exemplary embodiment;

FIG. 4 illustrates the arrangement of the features of the invention according to a preferred embodiment, for performing experiments;

FIG. 5 illustrates a graphic of an In-Oil sensor test wherein the Hydrogen H2 levels retrieved by said sensor is shown; and

FIG. 6 illustrates a graphic of a further In-Oil sensor test, wherein the Hydrogen H2 sensing target was set to 438.5 PPM in the gas space, and the Hydrogen H2 levels by hour are shown.

### DETAILED DESCRIPTION OF THE INVENTION

The different aspects of the present invention refer to an enhanced N₂ regulation system by integrating it with a H₂ sensor together as a combined package.

In a preferred embodiment the complete system comprises a Nitrogen gas cylinder, a 3-stage pressure-reducing regulator, high and low-pressure gauges, high and low-pressure alarm switches, an automatic pressure/vacuum-relief valve, a supply line with an isolation valve, a hydrogen sensor, and mechanical connections to the return line and the isolation valve of the Nitrogen system, along with a power supply and circuit breaker. This system is designed to detect the generation of hydrogen within a transformer, directly within the nitrogen headspace of the transformer.

In another preferred embodiment, the Nitrogen regulator is specifically engineered for use on transformer tanks, or any other electrical device requiring a clean and dry nitrogen blanket within the headspace above the insulating fluid, among others.

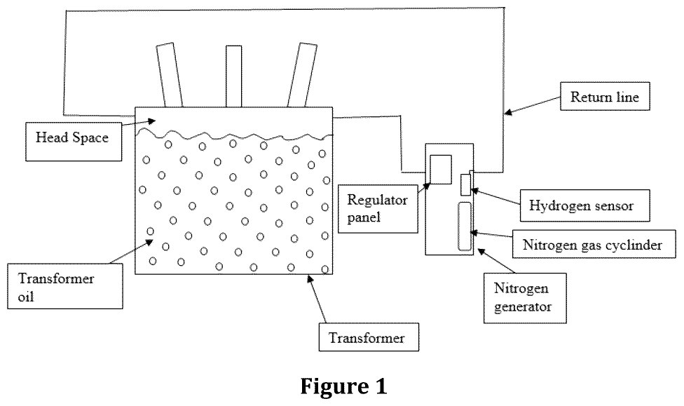

The present invention ensures a system's operation that maintains the set pressure range by either adding nitrogen or venting overpressure to the atmosphere as required. The final stage of the N₂ regulator system is responsible for maintaining the gas-blanketed tank pressure within the range of 0.5 to 5.0 psi consistently. If the tank pressure drops below the minimum of 0.5 psi, nitrogen is supplied from the storage tank through the regulator assembly to the transformer tank. In case the tank pressure rises above 5.5 psi, a relief valve will open to release excess pressure into the atmosphere, as depicted in FIG. 1.

In a preferred embodiment, a ¼″ non-corrosive line is connected from the transformer tank's headspace to the N₂ regulator system's supply line, to provide the transformer with a nitrogen blanket.

In another preferred embodiment, a second ¼″ non-corrosive line connects from the transformer headspace back to the return valve of the N₂ regulator. The opening of this valve allows for the purging of the transformer tank's headspace from ground level.

In a preferred embodiment, the hydrogen sensor is installed on the tank pressure side of the return valve to continuously monitor the headspace for the presence of hydrogen gas.

Furthermore, in a preferred embodiment, the hydrogen sensor is configurable to monitor the presence of hydrogen gas in a range between 0 to 100,000 PPM.

These positive pressure nitrogen gas regulating systems are instrumental in safeguarding transformer oil within the main tank against exposure to both oxidation and moisture, thereby maintaining the highest quality of insulating oil. It's widely acknowledged in the utility industry that hydrogen is produced due to internal faults exceeding 100° C. within a transformer. Hydrogen sensor technology can effectively monitor and track hydrogen generation within a transformer, either via the headspace (nitrogen blanket) or directly within the oil.

In a preferred embodiment, current invention comprises:
A) at least one gas cylinder (10), which serves as a fundamental component of the nitrogen regulation system integrated with the hydrogen sensor. This gas cylinder (10) is specifically designed to store high-purity nitrogen (N₂) under pressure, thereby maintaining an adequate nitrogen blanket within the transformer headspace. The inclusion of at least one gas cylinder (10) within the integrated nitrogen regulation system enhances the operational reliability of transformers. By ensuring a continuous supply of nitrogen gas, the gas cylinder (10) plays a vital role in safeguarding the integrity of the transformer and minimizing the risk of failure due to environmental factors.

The gas cylinder (10) is manufactured from durable materials, selected from a group comprising aluminum alloys, carbon fiber composites, or other high-strength materials, ensuring both robustness and resistance to corrosion. The cylinder features a cylindrical structure with a diameter ranging from 4 to 12 inches and a height that varies according to storage requirements, typically between 20 and 50 inches.

The upper portion of the gas cylinder (10) is equipped with a high-pressure valve (11), which allows for the controlled release of nitrogen gas into the nitrogen regulation system. This valve (11) is designed to meet safety standards, incorporating a pressure relief mechanism to prevent over-pressurization. Furthermore, a level gauge (12) is integrated into the cylinder, allowing for real-time monitoring of the nitrogen gas volume, thus facilitating efficient maintenance scheduling without the need for external measuring devices.

In an embodiment, the gas cylinder (10) is connected to the pressure-reducing regulator via a high-pressure supply line (13). This supply line is constructed from non-corrosive materials, such as stainless steel or reinforced polymers, ensuring compatibility with nitrogen gas and preventing any potential chemical reactions. The connection is designed to minimize turbulence in the gas flow, promoting a stable and reliable delivery of nitrogen gas to the transformer.

Operationally, when the nitrogen pressure within the transformer tank falls below a specified threshold, the pressure-reducing regulator (14) activates the valve (11) of the gas cylinder (10), allowing nitrogen to flow into the transformer headspace. This automatic response is crucial for maintaining optimal operating conditions, preventing the entry of moisture and oxidation that could compromise the quality of the transformer oil.

Moreover, the design of the gas cylinder (10) facilitates the replacement or refilling. The incorporation of quick-connect fittings allows for rapid exchanges of empty cylinders, enabling maintenance personnel to replenish the nitrogen supply efficiently without disrupting the transformer's operation.

In an embodiment, the at least one cylinder contains Nitrogen.
B) at least one pressure-reducing regulator (20). This regulator serves to modulate the pressure of nitrogen gas supplied from the gas cylinder to the transformer tank, ensuring optimal operating conditions while preventing the occurrence of overpressure or under pressure scenarios that could compromise the transformer's functionality.

The at least one pressure-reducing regulator not only stabilizes the pressure of the nitrogen blanket but also integrates seamlessly with the hydrogen sensor and other components of the system. This collaboration enhances the overall functionality and reliability of the transformer's operation, thereby contributing significantly to predictive maintenance and early fault detection

The pressure-reducing regulator is engineered to maintain a precise pressure range between 0.5 psi and 5.0 psi within the transformer's nitrogen headspace. It functions through a multi-stage reduction process, which involves the gradual decrease of the input gas pressure from the nitrogen gas cylinder to the desired output pressure. This is accomplished via an internal diaphragm mechanism that responds to fluctuations in the downstream pressure, thereby automatically adjusting the flow of nitrogen as needed.

In a preferred embodiment, the pressure-reducing regulator is equipped with high and low-pressure gauges that provide real-time monitoring of the nitrogen pressure levels within the system. These gauges are strategically positioned to ensure visibility for maintenance personnel, facilitating timely interventions when pressure levels deviate from the set parameters. Additionally, the regulator includes integrated alarm switches that activate when either high or low-pressure thresholds are breached, issuing warnings to prevent potential transformer failures.

Moreover, the design of the pressure-reducing regulator includes a built-in pressure/vacuum-relief valve. This safety feature is crucial in venting excess pressure to the atmosphere in cases where the pressure exceeds the upper limit of 5.5 psi. Such a mechanism ensures that the system operates within safe parameters, effectively mitigating risks associated with pressure buildup, which can lead to gas leaks or catastrophic transformer failures.

The construction materials utilized for the pressure-reducing regulator are selected for their durability and resistance to corrosion, particularly when in contact with nitrogen and potential contaminants present in the transformer oil. Commonly employed materials may include brass, stainless steel, and high-density polymers, which are capable of withstanding the operational stresses associated with gas flow regulation.

In an embodiment, the at least pressure-reducing regulator is a 3-stage pressure-reducing regulator.
C) at least one high-pressure gauge (22) and at least one low-pressure gauge (23), strategically configured to provide accurate monitoring of pressure levels within the nitrogen-blanketed transformer. The gauges (22, 23) are designed to operate seamlessly within the nitrogen regulation system, ensuring optimal performance and safety.

The incorporation of at least one high-pressure gauge (22) and one low-pressure gauge (23) within the integrated gas sensing and regulation system represents a significant advancement in the monitoring capabilities of nitrogen-blanketed transformers. By delivering accurate, real-time pressure readings, these gauges enhance the operational reliability of the transformer, ensuring its efficient and safe operation over time.

The high-pressure gauge (22) is specifically engineered to measure pressures in the range of 0 to 10 psi, allowing for precise detection of elevated pressure levels that may indicate potential gas buildup or system irregularities. In contrast, the low-pressure gauge (23) operates within a range of 0 to 1 psi, which is critical for identifying drops in pressure. Notably, if the pressure reading falls below the minimum threshold of 0.5 psi, the low-pressure gauge (23) activates an alert, prompting the nitrogen system to initiate gas supply from the nitrogen cylinder to maintain the necessary pressure within the transformer tank.

Both gauges (22, 23) are constructed from robust, corrosion-resistant materials, ensuring durability and reliability even under challenging operational conditions commonly encountered in transformer environments. The gauges feature clear, easy-to-read displays—either analog or digital—that facilitate quick visual assessments of pressure levels for operators. Additionally, each gauge is equipped with integrated alarm switches that trigger if pressure readings exceed specified limits, providing immediate notifications to maintenance personnel to address any potential issues before they escalate.

The high and low-pressure gauges (22, 23) are mounted at optimal locations within the nitrogen regulation system to allow for continuous monitoring of the gas-blanketed tank's pressure. This strategic positioning enhances the system's overall efficiency and reliability, contributing to proactive maintenance actions that can mitigate the risk of transformer failures.

Moreover, the installation of the high and low-pressure gauges (22, 23) is designed for simplicity, requiring no extensive modifications to the existing nitrogen regulation system. Their user-friendly interface allows for straightforward calibration and minimal maintenance, providing peace of mind to operators regarding the system's performance.
D) At least one high and low-pressure alarm switches; the integrated gas sensing and regulation system according to an embodiment of current invention incorporates at least one high and low-pressure alarm switch (30), designed to monitor and ensure optimal pressure conditions within the nitrogen-blanketed transformer environment. The configuration of these alarm switches is integral to maintaining the operational integrity of the transformer, thereby preventing potential failures that could arise due to pressure anomalies.

The inclusion of at least one high and low-pressure alarm switch (30) significantly enhances the functionality and safety of the integrated gas sensing and regulation system. By ensuring that the transformer operates within safe pressure limits at all times, these alarm switches provide peace of mind to operators and contribute to the overall longevity and reliability of the transformer system. The proactive nature of these alarm mechanisms not only safeguards the physical components of the transformer but also enhances operational efficiency, ultimately leading to a more effective maintenance strategy and reduced operational costs.

The high-pressure alarm switch (30a) is strategically positioned within the nitrogen regulation system to activate when the internal pressure of the transformer exceeds a critical threshold of 5.5 psi. This switch features a calibrated pressure sensor capable of detecting even slight variations in pressure levels. Upon reaching the predetermined limit, the alarm switch (30a) triggers an audible alert, which can be a loud siren or a beep, in addition to a visual signal, such as a flashing light. These alerts serve as immediate warnings to operators, indicating the presence of an overpressure condition that necessitates urgent attention.

The primary function of the high-pressure alarm switch (30a) is to mitigate the risk of catastrophic transformer failures caused by excessive internal pressure. Such failures can result in severe damage to the transformer, leading to costly repairs and prolonged downtime. By providing an early warning system, the high-pressure alarm switch (30a) enhances the safety profile of the transformer, allowing operators to take prompt corrective actions, such as venting excess nitrogen gas through the relief valve, thus maintaining safe operating conditions.

Conversely, the low-pressure alarm switch (30b) is engineered to detect conditions in which the pressure within the transformer drops below a critical level of 0.5 psi. This switch plays a crucial role in ensuring the integrity of the nitrogen blanket, which serves to protect the transformer oil from oxidation and moisture contamination. Upon detection of the low-pressure condition, the alarm switch (30b) activates, emitting a distinctive alarm to alert personnel of the insufficient nitrogen blanket. This alert prompts immediate investigation and corrective measures, such as replenishing nitrogen gas from the storage tank to restore optimal pressure levels.

The design of both alarm switches (30) facilitates seamless integration into the overall nitrogen regulation system, requiring no additional fittings or hardware for installation. This characteristic not only simplifies the setup process but also reduces associated costs, making the system more accessible to a broader range of applications. Furthermore, the alarm switches (30) are constructed from robust and durable materials, ensuring their ability to withstand the operational conditions within the transformer, such as fluctuations in temperature and pressure, thereby providing long-lasting reliability and performance.

To enhance operational efficiency, the alarm switches (30) can be integrated into a remote monitoring system. This allows for real-time data logging and analysis, empowering operators with the ability to track pressure trends over time. Such a capability is invaluable for predictive maintenance strategies, enabling the identification of potential issues before they escalate into critical failures.

The remote monitoring system can store up to one year of historical pressure data, facilitating trend analysis and enabling operators to make informed decisions regarding maintenance schedules. With the ability to access this data remotely, maintenance personnel can monitor the system from any location, significantly improving response times to potential issues.
E) at least one pressure/vacuum-relief valve (40), wherein the valve is strategically positioned within the nitrogen blanket assembly to ensure optimal pressure management and enhance the safety of the transformer under varying operational conditions.

The inclusion of at least one pressure/vacuum-relief valve (40) within the nitrogen regulation system not only optimizes the performance of the transformer but also significantly enhances its operational safety and efficiency. This innovative design element embodies the invention's commitment to providing a reliable, cost-effective solution for transformer maintenance and operation.

The pressure/vacuum-relief valve (40) is constructed from high-quality, corrosion-resistant materials, ensuring durability in the demanding environment of transformer operations. The valve features a compact design that allows for seamless integration into the nitrogen supply lines, with mechanical connections that facilitate straightforward installation and maintenance procedures.

Specifically, the valve (40) is Engineered to withstand high pressures, the housing of the valve is designed to resist corrosion and degradation caused by the transformer environment, ensuring longevity.

Furthermore, in a preferred embodiment the valve (40) comprises a precision-engineered sealing mechanism that guarantees a tight closure during normal operating conditions, preventing the unwanted escape of nitrogen gas.

In a preferred embodiment the valve (40) is equipped with an adjustable spring mechanism that allows operators to calibrate the pressure settings according to the specific requirements of the transformer application.

The primary function of the pressure/vacuum-relief valve (40) is to regulate the internal pressure within the transformer effectively. In scenarios where the internal pressure exceeds a predetermined threshold, specifically when it surpasses 5.5 psi, the valve automatically activates and opens. This action allows excess nitrogen gas to vent safely into the atmosphere, thereby preventing potential over-pressurization of the transformer tank, which could lead to catastrophic failures.

Conversely, the valve is also designed to address vacuum conditions. In instances where the internal pressure drops below 0.5 psi, the valve prevents atmospheric air from infiltrating the transformer tank. This functionality is critical for maintaining the integrity of the nitrogen blanket, which serves to protect the transformer oil from moisture ingress and oxidation, thus preserving its dielectric properties.

The pressure/vacuum-relief valve (40) incorporates several innovative features that enhance its performance and reliability; as a non-limiting example, the valve (40) includes: an adjustable pressure setting that can be tailored to accommodate the specific pressure requirements of different transformer models, ensuring optimal functionality; integrated visual indicators are present on the valve, providing real-time feedback regarding its operational status allowing operators to quickly assess whether the valve is functioning correctly or if maintenance is needed; built-in safety mechanisms that prevent inadvertent operation, such as a locking feature that secures the valve in its closed position during routine maintenance; a lightweight structure that allows for simple handling and installation. Additionally, the maintenance access points are strategically placed to facilitate quick inspections and servicing, combinations thereof and other similar features.

The integration of the pressure/vacuum-relief valve (40) significantly enhances the overall safety and reliability of the transformer. By preventing both over-pressurization and vacuum conditions, this component contributes to increase the transformer longevity by mitigating mechanical stress on the transformer structure and associated components, the valve helps extend the service life of the transformer, so as enhanced safety protocols by reducing the likelihood of catastrophic failures that could result from pressure imbalances, thereby improving operational safety for personnel and equipment; the cost-effective maintenance based on the reliable operation of the valve lowers the frequency of maintenance checks and interventions, translating to reduced operational costs over time; compliance with industry standards, ensuring compliance with industry safety regulations, making it a crucial element in the transformer's overall safety strategy.
F) at least one supply line with an isolation valve (58) ensures an efficient and controlled flow of gas. This supply line (58) is specifically engineered to integrate into the nitrogen regulation system, enhancing the overall performance and safety of the transformer operation.

The supply line (58) with the isolation valve (70) contribute to the reliability and safety of transformer operations. By ensuring efficient nitrogen delivery, facilitating easy maintenance access, and incorporating robust safety features, this component plays an integral role in safeguarding the performance and longevity of the transformer system.

In a preferred embodiment, the supply line (58) is constructed from a robust, non-corrosive material such as stainless steel or high-density polyethylene (HDPE). These materials are selected for their superior durability and resistance to corrosion, especially in the challenging environmental conditions often encountered in transformer applications. This selection not only guarantees the integrity of the supply line over time but also minimizes the risk of gas leaks, thereby ensuring consistent and reliable gas delivery to the transformer tank (65).

The supply line (58) features a carefully designed isolation valve (70) strategically positioned along its length. The isolation valve (70) serves multiple purposes, primarily providing a means to swiftly shut off the gas flow to the transformer tank (65) during maintenance operations or in emergency situations. This design feature is critical, as it allows for safe servicing of the nitrogen system without the risk of nitrogen gas leaks or exposure to the surrounding environment, thus safeguarding both personnel and equipment.

The isolation valve (70) is preferably a quarter-turn ball valve, chosen for its simplicity and efficiency in operation. This type of valve allows for quick and effortless opening or closing of the gas flow, enabling operators to swiftly cut off the nitrogen supply when necessary. The user-friendly design minimizes the potential for human error, enhancing operational safety. Additionally, the valve is equipped with a visual indicator that clearly displays whether the valve is in the open or closed position, ensuring that users are always fully aware of the system status.

To ensure ease of installation and maintenance, the supply line (58) with the isolation valve (70) requires minimal fittings, streamlining the overall installation process and reducing costs associated with additional hardware. The design allows for straightforward integration into existing systems, making retrofitting an uncomplicated endeavor that does not necessitate extensive modifications to current infrastructure.

Furthermore, the isolation valve (70) is equipped with advanced sealing mechanisms, such as high-performance O-rings and gaskets, to ensure a leak-proof connection at all junctions. This feature is essential for enhancing the safety of the system by preventing nitrogen gas from escaping into the environment and ensuring that the transformer maintains its required nitrogen blanket effectively.

In an additional embodiment, the supply line (58) may incorporate sensors or pressure gauges to monitor the nitrogen flow rate and pressure within the system. This integration provides real-time data, allowing operators to make informed decisions regarding the maintenance and operation of the nitrogen regulation system. The inclusion of such monitoring devices contributes to the predictive maintenance capabilities of the overall system, aligning with the invention's goal of preventing transformer failures and facilitating timely interventions.
G) at least one hydrogen sensor (80), which is engineered to detect the concentration of hydrogen gas (H₂) present in the headspace of the transformer, which is essential for monitoring the condition of the transformer oil and for early detection of potential faults. By integrating the hydrogen sensor into the nitrogen system, the invention offers a streamlined solution that enhances both safety and efficiency.

The at least one hydrogen sensor integrated within the nitrogen regulation system is a sophisticated and essential component designed to ensure the optimal functioning of nitrogen-blanketed transformers. By offering a robust solution for monitoring and maintaining transformer health while minimizing operational costs, the hydrogen sensor contributes significantly to the reliability and safety of transformer operations. The invention not only addresses the immediate concerns related to hydrogen detection but also enhances the overall maintenance strategy, providing a comprehensive approach to transformer health management.

The hydrogen sensor is constructed from durable, non-corrosive materials, such as high-grade stainless steel or specialized polymers, ensuring its longevity and effectiveness in the harsh conditions present within transformer operations. The sensor features a compact design, allowing for seamless integration into existing transformer setups without the need for extensive modifications. The form factor is specifically optimized for installation within the nitrogen headspace, ensuring accurate gas measurements while minimizing space requirements.

The sensor operates on advanced electrochemical or semiconductor technology, depending on the specific embodiment utilized. This technology enables the sensor to accurately measure hydrogen concentrations ranging from 0 to 100,000 parts per million (PPM). The operation is based on the principle of detecting changes in electrical resistance or voltage that occur in the presence of hydrogen gas. This provides real-time data on the gas levels, allowing for immediate analysis and response.

An innovative feature of the hydrogen sensor is its self-calibrating mechanism, which minimizes maintenance requirements and enhances reliability. This feature allows the sensor to adjust its sensitivity and response based on the prevailing conditions, thus ensuring consistent and accurate readings without the need for manual intervention. The self-calibration process is initiated automatically at predetermined intervals or during specific operational conditions, ensuring optimal performance over the sensor's lifespan.

In a preferred embodiment, the hydrogen sensor is equipped with the capability to log data and provide real-time monitoring. It can store up to one year of historical data, facilitating trend analysis and predictive maintenance. This remote access feature allows operators to monitor hydrogen levels without being physically present, thereby enhancing operational efficiency and safety. The logged data can be transmitted via wireless communication protocols, enabling easy access through mobile devices or centralized control systems.

In an embodiment, the Hydrogen (H₂) sensor is mounted inside the nitrogen panel; this is a feature not-previously known in the state of art, since no other disclosure referred to a nitrogen panel with an integrally-formed/mounted sensor.

In an embodiment, the nitrogen panel of current application is installed outside the transformer. With this possibility, the user or customer only must mount or adapt the nitrogen panel (current invention) to a transformer and the invention will sense the amount of hydrogen in the transformer.

Bearing the above in mind, the first gas that is detected in the transformer is the hydrogen; it is known that high or Critical level of hydrogen (along with other gases) cause that the transformer would be taken out of service to maintenance

Thus, the present invention is directed to monitoring and detecting when high levels of hydrogen (0-50,000 ppm) form inside the monitored transformer. Once the present invention detects said critical levels or detects that the level of hydrogen is too high and continues to increase (along with other gases), the invention notifies the customer, which could determinate that the transformer would need maintenance before the transformer suffer any significant damage.

On the other hand, when the levels of hydrogen are within the threshold, the user could determinate that the transformer is operating in a normal condition.

By continuously monitoring hydrogen levels, the sensor plays a vital role in the preventive maintenance strategy for transformers. Hydrogen generation is indicative of internal faults, often resulting from temperatures exceeding 100° C. The early detection of hydrogen presence enables prompt intervention, reducing the risk of catastrophic transformer failures and associated downtime. Furthermore, the sensor is designed to trigger alarms or notifications when gas concentrations exceed predefined thresholds, ensuring that operators can take timely action to mitigate risks.

The hydrogen sensor is operatively connected to a user-friendly interface that displays real-time readings, historical data trends, and alerts. This interface is designed to be intuitive, allowing operators to easily navigate through various functions, adjust settings, and review data logs. In addition, the sensor can be integrated into existing monitoring systems, enhancing the overall capabilities of transformer management solutions.

The hydrogen sensor is designed for minimal maintenance and has an extended operational lifespan of over 10 years, making it a cost-effective solution for transformer monitoring. Regular maintenance checks can be performed to ensure optimal performance, including visual inspections and functionality tests. The durable construction and protective housing ensure that the sensor remains operational even under extreme environmental conditions, including temperature fluctuations and exposure to moisture.

In a preferred embodiment, the at least one hydrogen (H₂) sensor is configured to detect the generation of hydrogen within a transformer, directly within the nitrogen headspace of the transformer.
H) at least one power supply (95a) and at least one circuit breaker (95b); in an exemplary embodiment, said at least one power supply (95a) and at least one circuit breaker (95b) are incorporated within the integrated gas sensing and regulation system and is designed to ensure the reliable operation of the hydrogen sensor and nitrogen regulation system.

The power supply (95a) is configured to provide a stable electrical output required for the optimal functioning of the sensor and associated electronic components, safeguarding against power fluctuations that could adversely affect performance and lead to incorrect readings or system malfunctions.

The power supply (95a) can be selected from various options, including but not limited to, alternating current (AC) adapters, direct current (DC) power supplies, or renewable energy solutions such as solar panels, depending on the installation environment and operational requirements of the transformer system. In a preferred embodiment, the power supply (95a) is designed to operate within a voltage range of 12V to 24V, with sufficient amperage of at least 5 A to support all connected devices, ensuring consistent operation without interruption and minimizing the risk of power-related failures.

Furthermore, the system is equipped with at least one circuit breaker (95b), strategically placed to enhance safety and protect against overload or short circuits. This circuit breaker (95b) serves as a critical protective measure that automatically disconnects the power supply (95a) in the event of an electrical fault, thereby preventing potential damage to the system components, including the hydrogen sensor and the nitrogen regulation apparatus, and ensuring the safety of personnel working in proximity to the transformer.

The circuit breaker (95b) is designed to be resettable, allowing for easy restoration of power once the fault condition has been addressed. It is rated for appropriate current levels based on the overall system requirements, with a trip rating calibrated to safeguard the components without unnecessary interruptions to system operation. For example, the circuit breaker (95b) may have a trip current rating of 10 A to 15 A, suitable for the anticipated load while providing a margin of safety.

In an additional preferred embodiment, both the power supply (95a) and the circuit breaker (95b) are housed within a protective enclosure (95c) that is resistant to environmental factors such as moisture, dust, and vibrations, ensuring durability and long-term reliability in various operational conditions. This enclosure (95c) is equipped with accessible connection points for easy maintenance and troubleshooting, further enhancing the overall user experience.

Moreover, the protective enclosure (95c) may also feature indicators such as LED lights to provide real-time feedback on the operational status of the power supply (95a) and circuit breaker (95b). These indicators can inform users of normal operation, faults, or maintenance needs, thereby improving system oversight and proactive maintenance efforts.

The integration of the power supply (95a) and circuit breaker (95b) within the nitrogen gas sensing and regulation system not only facilitates effective operation but also ensures adherence to safety standards essential in high-stakes environments such as transformer operations. This comprehensive approach to safety and reliability provides peace of mind to users regarding the integrity of the system, enabling them to focus on maintenance and operational efficiency without the constant concern of electrical failures impacting transformer performance.

In an embodiment, the regulation system is adapted in a cavity or is adapted in the inner portion of a transformer by mechanical means and/or by magnetic means.

In this sense, for “mechanical means and/or magnetic means” it should be understood that the system, in an embodiment, comprise an adaptable platform which allows to lock the system in the transformer via screws, bolts, or any other mechanical known and commercial available medium; in a further embodiment, the system comprise an adaptable platform comprising magnets or any other magnetic source, which allows to lock the system in the transformer body.

The nitrogen regulation system consists of a nitrogen gas supply cylinder with its own control valve, a supply pressure gauge, a three-stage pressure reducing assembly and the piping and valves that control the flow of gas to and from the tank. The cylinder could be used as a standard, commercially available, 244 cu. ft. nitrogen gas cylinder pressurized to 2000 psi.

[symbol] ; nevertheless, any other cylinder could be used and be within the scope, spirit and essence of current application. Replacement cylinders should meet all required pressure vessel specifications and be filled with oil pumped nitrogen or nitrogen with a certified moisture content of less than 0.03 percent by weight. The impurity content must be less than 7.5 parts per million.

Nitrogen consumption is dependent on transformer load variations and on the condition of the gas pressurizing equipment. The cylinder must be replaced when the supply pressure gauge reads 200 psi or below. The factory set point for the low bottle alarm is 200 PSIG. The system includes electrical connection points for low gas supply, high tank pressure and low tank pressure alarms. When the nitrogen regulation system is correctly set-up and operating, transformer tank pressure will maintain at 0.5 psi minimum and 5.0 psi maximum. During periods of transformer cooling, the overall tank pressure will decrease. If the tank pressure drops below 0.5 psi, nitrogen gas flows from the bottle supply cylinder through the reducing valve assembly and into the tank until the 0.5 psi pressure is restored. During periods of transformer heating, tank pressure will increase. If tank pressure exceeds 5.0 psi, the regulator assembly will vent the excess nitrogen to atmosphere to prevent tank damage or PRD operation. The 3rd stage regulator supplying nitrogen to the transformer tank has an adjustable range of 0-2 psi and is set to a slight positive pressure (0.5 psi standard) at the factory. A 0.5 psi nitrogen supply pressure and a relief valve breaking pressure of 5.0 psi are chosen in order to provide a 4.5 psi tank regulation band. Increasing the nitrogen supply pressure will decrease the regulation band for the transformer tank and may increase nitrogen use during periods of heavy thermal cycling.

Adjustable alarm contacts are provided to indicate max/min tank pressures selected by user. Typical alarm points would be set just outside of the selected regulation band. For example, user alarms are normally recommended to be set for 0.2 psi and 5.5 psi for a 0.5 to 5.0 regulation band.

The compound pressure gauge monitors the gas pressure in the transformer tank. The gauge has a range from negative 15.0 psi to positive 15.0 psi (−15.0 psi to +15.0 psi) and is equipped with two adjustable alarm contacts. The gauge is shipped with both moveable alarms adjusted to zero to prevent shipping damage due to shipping vibration. Before placing the unit in service, these alarm points must be adjusted to be outside of the normal regulation band.

### Factory Recommended Settings Would be 0.2 psi and 5.5 psi

During normal operations, the pressure on the transformer tank is expected to fluctuate between 0.5 psi and 5.0 psi, following the temperature variations in the transformer, at which both SW #1 contacts will stay open. In the event the pressure drops to 0.2 psi or below (the low pressure alarm setting), the SW #1 low pressure contact (Red-White) will close, sending an alarm, if monitored. On the other hand, if the pressure on the transformer tank increases to 5.5 psi or above (the high-pressure alarm setting), the SW #1 high pressure contact will close, sending an alarm, if monitored. The Alarm Enable Switch is used to disable alarm signals during a nitrogen bottle change. The three-stage pressure reducer assembly regulates the flow of gas from the supply cylinder to the transformer tank. Stage one reduces the pressure of the gas flowing from the supply cylinder from 2200 to 100 psi. The regulator at stage two reduces the pressure of the gas flowing from 100 to 7 psi.

The third stage reduces the pressure of the gas flowing from 7 to 0.5 psi (adjustable 0-2 psi) and controls the flow of gas to the tank, admitting gas into the tank whenever the tank pressure drops below 0.5 psi. The third stage also includes a pressure vacuum device, which opens if tank pressure rises beyond 5.0 psi or a vacuum below 3.0 psi. P-V adjustable range is 3-12 psig. The three stages are factory set.

Many modifications and other embodiments of the invention will come to mind to one skilled in the art to which the invention pertains, who has the benefit of the teachings presented in the foregoing descriptions and the associated drawings. Therefore, it should be understood that the invention is not to be limited to the specific and exemplary embodiments described, but rather that modifications and other embodiments are intended to be included within the scope of the appended claims. Although specific terms are used herein, they are used only in a generic and descriptive sense and not for limiting purposes. Likewise, it must be understood that the raw materials with which the different components comprising the invention described in this document can be manufactured, and other elements may vary without departing from the scope and spirit of the invention and, therefore, the referred modalities should not consider limiting.

### EXAMPLES

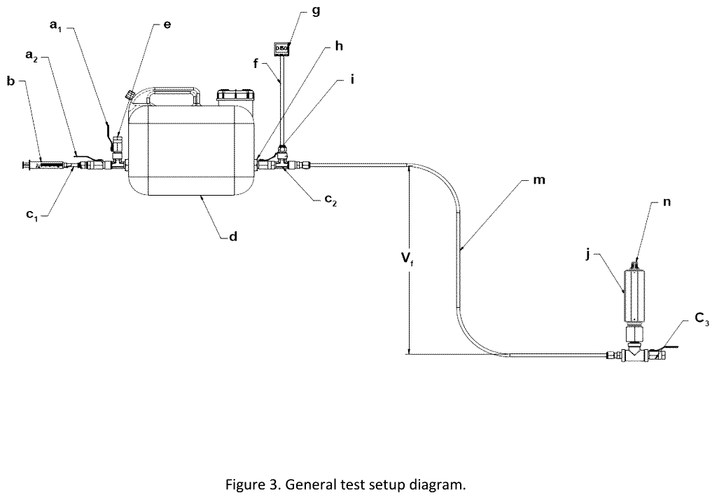

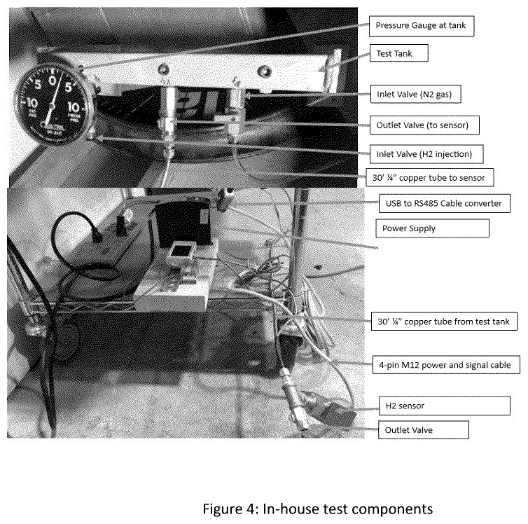

As seen in FIGS. 3 and 4, a controlled test was performed to verify the feasibility of sensing hydrogen gas in transformer tank and rate at which hydrogen gas is diffused. Test was conducted under static pressure, with hydrogen sensor installed on return line of the copper tube from the test tank.

The test method to detect H2 gas in a transformer according to the present invention as shown in FIG. 3, which illustrates a general test setup diagram showing the features of the present invention according to an exemplary embodiment, said setup comprises:
a₁—which according to an exemplary embodiment of the present invention, said “a₁” is a first valve;
a₂—which according to an exemplary embodiment of the present invention, said “a₂” is a second valve;
b—which according to an exemplary embodiment of the present invention, said “b” is a syringe with a volume capacity in a range of 1 to 10 ml;
c₁—which according to an exemplary embodiment of the present invention, said “c₁” is a first connecting tube configured to operatively connect the syringe with the tank (d);
c₂—which according to an exemplary embodiment of the present invention, said “C₂” is a second connecting tube configured to operatively connect the tank (d) with the hydrogen sensor (j);
c₃—which according to an exemplary embodiment of the present invention, said “C₃” is third valve disposed of in an outlet to the hydrogen sensor (j);
d—which according to an exemplary embodiment of the present invention, said “d” is a reservoir or a tank with a capacity in a range of 5000 to 15,000 mL;
e—which according to an exemplary embodiment of the present invention, said “e” is a port to charge the tank (d) with nitrogen, being said charge of nitrogen in a range of 0.1 to 1 PSI;
f—which according to an exemplary embodiment of the present invention, said “f” is a tubing configured to allow the flow of gas to the digital pressure gauge;
g—which according to an exemplary embodiment of the present invention, said “g” is a digital press gauge;
h—which according to an exemplary embodiment of the present invention, said “h” is at least one bulkhead disposed in the outlet of the tank (d), and operatively connected to the second connecting tube (c₂);
i—which according to an exemplary embodiment of the present invention, said “i” is a third valve operatively connected to the tubing configured to allow the flow of gas to the hydrogen sensor (f);
v_{f}—which according to an exemplary embodiment of the present invention, said “v_{f}” is a vertical distance measured from the outlet of the tank (d) and the inlet of the hydrogen sensor (j), being said vertical distance in a range of 5 to 10 feet.
j—which according to an exemplary embodiment of the present invention, said “j” is a hydrogen sensor, which is fed from the tank (d) and the tubing configured to allow the flow of hydrogen gas (f) throughout a tubing (m);
m—which according to an exemplary embodiment of the present invention, said “m” is a tubing configured to feed the hydrogen sensor (j) from the tank (d) and the tubing configured to allow the flow of gas (f); and
n—which according to an exemplary embodiment of the present invention, said “n” is a m12 cable connector for power and signal.

Bearing the above in mind, the method according to the present invention and according to an embodiment thereof comprises the steps of:
i) close valve a₂ and maintain valves “h” and “C₃” open;
ii) charge and purge the tank (d) through valve “a₁” with N₂ gas;
iii) close valve “C₃” to contain the N₂ gas charged in the previous step;
iv) monitor the tank (d) and test line pressure with the digital gauge (g);
v) start the hydrogen sensor (j) and close valve “a₁” isolate the tank with the N₂ gas charged in step ii) and close valve “i” to isolate the N₂ gas into the tubing (m);
vi) monitor the test line pressure with the digital gauge (g);
vii) connect to valve “a₂” the syringe (b) filed with H₂ gas;}
viii) open valve “a₂” and insert the H2 gas disposed in the syringe (b);
ix) close valve “a₂” after 3 to 8 minutes; and
x) wait from 10 to 20 minutes so that the H₂ gas disperses through the tank (d);
xi) open valve “i” so that the injected H₂ gas reaches the hydrogen sensor (j) and an be detected the presence of said H₂.

In an embodiment, within step iii) of the method, the charge and purge of the tank (d) is made with a range of 1 to 2 PSI of N₂ gas.

In an embodiment, within step vii) of the method, the syringe (b) is filed in a range of 1.8 to 3.20 ml of H₂ gas, in order to raise the tank (d) H₂ concentration to the ppm level in a range between 200 and 300 ppm.

Setup consisted of the following—tank, ¼″ copper tube (30 ft), 2 inlet valves, one outlet valve, pressure gauge at the tank, 24 VDC power supply, USB to RS485 adapter and 4-pin M12 cable.

Test tank was purged with N2 then held at static pressure of 0.5 psi nitrogen and no gas flowing through a closed system. Test tank elevated 30 ft above the sensor and output port of the tank connected to hydrogen sensor via 30 ft non-corrosive pipe. With port to the sensor closed, a preset level of 5 ml hydrogen gas was injected into test tank. After injecting and closing the valve to the tank, the output valve to the sensor was opened to let the hydrogen gas diffuse into the sensor via the tube.

The onboard memory of the hydrogen sensor recorded data every 5 seconds. No pressure drop was observed during the test. Data was downloaded from the sensor every 24 hrs to verify if target H2 ppm reached.

Target ppm of H2 gas was observed within 3 days during the first run.

To verify repeatability, the test was repeated 2 more times (one with 5 ml and another with 2.5 ml hydrogen gas). See conclusions section for the test results.

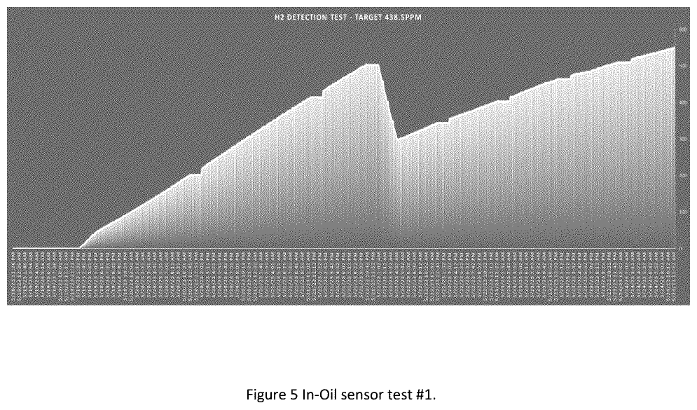

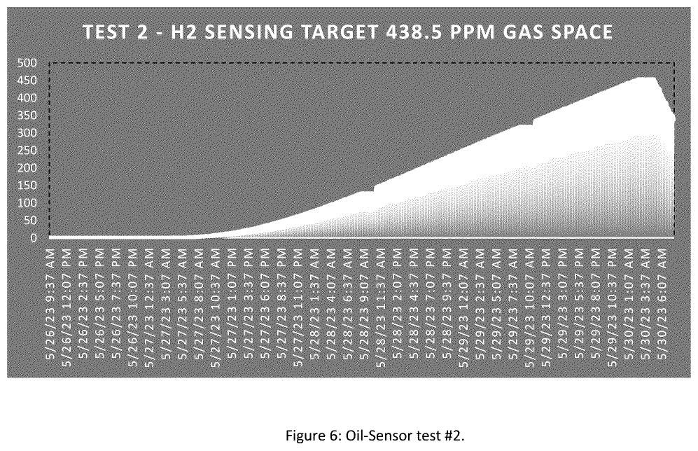

On this basis, referring to FIGS. 5 and 6, it was observed the rate at which sensor detects hydrogen gas is directly proportional to the volume of injected gas in the tank. With higher volume the gas will diffuse at a faster rate to the sensor.

(Note: For this test we used in-oil hydrogen sensor. If gas-space sensor is used, then H2 ppm data will be multiplied by 20)

Test #1 Target reached in 3 days (Trial #1). We continued to test repeatability.

Test #2 target reached in 4 days (Trial #2):
Type of sensor: Oil space hydrogen sensor
Data range: May 26, 2023-Jun. 2, 2023
Temperature range: 28° C.-31° C.
Detected volume range: 0-591 ppm.
Test target: 438.5 ppm (Gas space), 8770 ppm (Oil space)

Target volume reached within 4 days (Trial #1 reached within 3 days).

### CONCLUSION

The test results prove that hydrogen gas is detected in nitrogen blanketed tank doped with hydrogen. Hydrogen sensor successfully logged data every 5 seconds and no errors or interruptions were noted during the test. A higher volume of hydrogen gas at the same tank psi will diffuse to the sensor at a faster rate. See tabulated results below.

### TEST SUMMARY

| | | |
|---|---|---|
| Volume of H2 | | Target Reached |
| injected | Targeted Volume | (Total duration) |

| | | | | |
|---|---|---|---|---|
| 5 | ml | 438.5 | PPM | 3 days |
| 5 | ml | 438.5 | PPM | 4 days |
| 2.5 | ml | 225 | PPM | 7 days & 11 hours |

1. A monitoring system configured for regulate Nitrogen (N₂), the regulation system comprising:
at least one gas cylinder, wherein the gas cylinder contains Nitrogen,
at least one pressure-reducing regulator, being equipped with at least one high and low-pressure gauges,
at least one high and at least one low-pressure alarm switches,
at least one pressure/vacuum-relief valve,
at least one supply line with an isolation valve,
at least one hydrogen sensor, and
at least one power supply and at least one circuit breaker
wherein the monitoring system consists of:
a nitrogen gas supply cylinder with its own control valve, a supply pressure gauge, a three-stage pressure reducing assembly and the piping and valves that control the flow of gas to and from the tank;
electrical connection points for low gas supply, high tank pressure and low tank pressure alarms,
wherein during periods of transformer cooling the overall tank pressure will decrease nitrogen gas flows from the bottle supply cylinder through the reducing valve assembly and into the tank until the pressure is restored;
wherein during periods of transformer heating, tank pressure will increase, and the regulator assembly will vent the excess nitrogen to atmosphere to prevent tank damage or PRD operation;
a nitrogen supply pressure and a relief valve breaking pressure, being configurable to provide a tank regulation band;
adjustable alarm contacts, which are configurated to indicate max/min tank pressures selected by user;
wherein increasing the nitrogen supply pressure will decrease the regulation band for the transformer tank and may increase nitrogen use during periods of heavy thermal cycling,
the regulation system further comprises a Hydrogen (H₂) sensor configured to detect the generation of hydrogen within a transformer,
the hydrogen (H₂) sensor is disposed directly within the nitrogen panel,
the nitrogen panel is installed outside the transformer, and
wherein the hydrogen sensor monitors the nitrogen panel for the presence of hydrogen gas in quantities between 0-50,000 ppm.

2. The regulation system according to claim 1, wherein during normal operations:
the pressure on the transformer tank is expected to fluctuate between 0.5 psi and 5.0 psi, following the temperature variations in the transformer, at which both SW #1 contacts will stay open;
in the event the pressure drops to 0.2 psi or below (the low pressure alarm setting), the SW #1 low pressure contact (Red-White) will close, sending an alarm, if monitored;
if the pressure on the transformer tank increases to 5.5 psi or above (the high-pressure alarm setting), the SW #1 high pressure contact will close, sending an alarm, if monitored;
wherein the Alarm Enable Switch is used to disable alarm signals during a nitrogen bottle change
wherein the three-stage pressure reducer assembly regulates the flow of gas from the supply cylinder to the transformer tank;
stage one reduces the pressure of the gas flowing from the supply cylinder from 2200 to 100 psi; and
wherein the regulator at stage two reduces the pressure of the gas flowing from 100 to 7 psi and the third stage reduces the pressure of the gas flowing from 7 to 0.5 psi (adjustable 0-2 psi) and controls the flow of gas to the tank, admitting gas into the tank whenever the tank pressure drops below 0.5 psi.

3. The regulation system according to claim 1, wherein the pressure-reducing regulator is a 3-stage pressure-reducing regulator.

4. The regulation system according to claim 1, wherein the gas cylinder is configurable to store high-purity nitrogen (N2) under pressure, ensuring a continuous supply of nitrogen gas.

5. The regulation system according to claim 1, wherein the gas cylinder further comprises: a high-pressure valve in the upper portion of the gas cylinder, a level gauge inside the cylinder, a pressure-reducing regulator and wherein the cylinder is connected to the pressure-reducing regulator via a high-pressure supply line.

6. The regulation system according to claim 1, wherein the pressure-reducing regulator maintains a pressure range between 0.5 psi and 5.0 psi within the transformer's nitrogen headspace.

7. The regulation system according to claim 1, wherein the pressure-reducing regulator comprises a built-in pressure/vacuum-relief valve.

8. The regulation system according to claim 1, wherein the pressure-reducing regulator is manufactured in brass, stainless steel, and high-density polymers, which are capable of withstanding the operational stresses associated with gas flow regulation.

9. The regulation system according to claim 1, wherein the at least pressure-reducing regulator is a 3-stage pressure-reducing regulator.

10. The regulation system according to claim 1, wherein the high-pressure gauge measures pressures in the range of 0 to 10 psi and the low-pressure gauge measures pressures in the range of 0 to 1 psi.

11. The regulation system according to claim 1, wherein both high-pressure gauge and low-pressure gauge are equipped with integrated alarm switches that trigger if pressure readings exceed specified limits.

12. The regulation system according to claim 1, wherein the at least one high and low-pressure alarm switch is positioned within the nitrogen regulation system to activate when the internal pressure of the transformer exceeds a critical threshold of 5.5 psi.

13. The regulation system according to claim 1, wherein the at least one low-pressure alarm switch is configurable to detect conditions in which the pressure within the transformer drops below a critical level of 0.5 psi.

14. The regulation system according to claim 1, wherein the at least one pressure/vacuum-relief valve is positioned within the nitrogen blanket assembly.

15. The regulation system according to claim 1, wherein the at least one pressure/vacuum-relief valve comprises a precision-engineered sealing mechanism that guarantees a tight closure during normal operating condition preventing the unwanted escape of nitrogen gas.

16. The regulation system according to claim 1, wherein the at least one pressure/vacuum-relief valve comprises an adjustable spring mechanism that allows operators to calibrate the pressure settings.

17. The regulation system according to claim 1, wherein the at least one pressure/vacuum-relief valve comprises: an adjustable pressure setting; integrated visual indicators; built-in safety mechanisms; a structure and wherein the maintenance access points are placed to facilitate quick inspections and servicing, combinations thereof and other similar features.

18. The regulation system according to claim 1, wherein the at least one supply line with an isolation valve is manufactured from a robust, non-corrosive material such as stainless steel or high-density polyethylene (HDPE).

19. The regulation system according to claim 1, wherein the isolation valve is a quarter-turn ball valve.

20. The regulation system according to claim 1, wherein the isolation valve is equipped with sealing mechanisms, such as high-performance O-rings and gaskets.

21. The regulation system according to claim 1, wherein the supply line incorporates sensors or pressure gauges to monitor the nitrogen flow rate and pressure within the system.

22. The regulation system according to claim 1, wherein the hydrogen sensor operates on electrochemical or semiconductor technology to measure hydrogen concentrations ranging from 0 to 100,000 parts per million (PPM).

23. The regulation system according to claim 1, wherein the hydrogen sensor further comprise a self-calibrating mechanism.

24. The regulation system according to claim 1, wherein the hydrogen sensor is capable to log data and provide real-time monitoring, store up to one year of historical data, facilitating trend analysis and predictive maintenance.

25. The regulation system according to claim 1, wherein the nitrogen panel of current application is installed outside the transformer.

26. The regulation system according to claim 1, wherein the hydrogen sensor is operatively connected to a user-friendly interface that displays real-time readings, historical data trends, and alerts.

27. The regulation system according to claim 1, wherein the at least one power supply and at least one circuit breaker are incorporated within the integrated gas sensing and regulation system.

28. The regulation system according to claim 1, wherein the power supply can be selected from various options, including but not limited to, alternating current (AC) adapters, direct current (DC) power supplies, or renewable energy solutions such as solar panels, and wherein the power supply is designed to operate within a voltage range of 12V to 24V, with sufficient amperage of at least 5 A to support all connected devices.

29. The regulation system according to claim 1, wherein the circuit breaker has a trip current rating of 10 A to 15 A.

30. The regulation system according to claim 1, wherein both the power supply and the circuit breaker are housed within a protective enclosure that is resistant to environmental factors such as moisture, dust, and vibrations, ensuring durability and long-term reliability in various operational conditions.

31. The regulation system according to claim 1, wherein the system is contained within a cavity or could be adapted to the inner portion of a transformer.

32. The regulation system according to claim 1, wherein replacement cylinders meet all required pressure vessel specifications and be filled with oil pumped nitrogen or nitrogen with a certified moisture content of less than 0.03 percent by weight and wherein the impurity content must be less than 7.5 parts per million.

33. The regulation system according to claim 1, wherein:
the transformer tank pressure will maintain at 0.5 psi minimum and 5.0 psi maximum;
during periods of transformer cooling, the overall tank pressure will decrease below 0.5 psi, and the nitrogen gas flows from the bottle supply cylinder through the reducing valve assembly and into the tank until the 0.5 psi pressure is restored;
during periods of transformer heating, tank pressure will increase exceeding 5.0 psi, the regulator assembly will vent the excess nitrogen to atmosphere to prevent tank damage or PRD operation; the 3rd stage regulator supply nitrogen to the transformer tank has an adjustable range of 0-2 psi and is set to a slight positive pressure (0.5 psi standard); and
a 0.5 psi nitrogen supply pressure and a relief valve breaking pressure of 5.0 psi provides a 4.5 psi tank regulation band.

34. The regulation system according to claim 1, wherein the compound pressure gauge:
monitors the gas pressure in the transformer tank; and
has a range from negative 15.0 psi to positive 15.0 psi (−15.0 psi to +15.0 psi); and
is equipped with two adjustable alarm contacts.

35. The regulation system according to claim 2, wherein user alarms are normally recommended to be set for 0.2 psi and 5.5 psi for a 0.5 to 5.0 regulation band.

36. The regulation system according to claim 2, wherein the third stage also includes a pressure vacuum device, which opens if tank pressure rises beyond 5.0 psi or a vacuum below 3.0 psi. P-V adjustable range is 3-12 psig.

37. The regulation system according to claim 4, wherein the gas cylinder is manufactured from durable materials, selected from a group comprising aluminum alloys, carbon fiber composites, or other high-strength materials, ensuring both robustness and resistance to corrosion, with a diameter ranging from 4 to 12 inches and a height between 20 and 50 inches.

38. The regulation system according to claim 5, wherein the supply line is constructed from non-corrosive materials, such as stainless steel or reinforced polymers, ensuring compatibility with nitrogen gas and preventing any potential chemical reactions.

39. The regulation system according to claim 5, wherein the nitrogen pressure within the transformer tank falls below a specified threshold, the pressure-reducing regulator activates the valve of the gas cylinder, allowing nitrogen to flow into the transformer headspace.

40. The regulation system according to claim 7, wherein the built-in pressure/vacuum-relief valve ins configurable to vent excess pressure to the atmosphere in cases where the pressure exceeds the upper limit of 5.5 psi.

41. The regulation system according to claim 16, wherein the adjustable spring mechanism is configurable so that when the internal pressure exceeds 5.5 psi, the valve automatically activates and opens, and when the internal pressure drops below 0.5 psi, the valve prevents atmospheric air from infiltrating the transformer tank.

42. The regulation system according to claim 30, wherein the protective enclosure further comprise indicators such as LED lights to provide real-time feedback on the operational status of the power supply and circuit breaker.

43. The regulation system according to claim 31, wherein the system is adapted to the inner portion of a transformer by mechanical means and/or by magnetic means.
